(19) United States
(12) Patent Application Publication
(10) Pub. No.: US 2012/0020832 A1
(43) Pub. Date: Jan. 26, 2012

(54) SYSTEM AND METHOD FOR ABATEMENT OF ALLERGENS, PATHOGENS, AND VOLATILE ORGANIC COMPOUNDS

(75) Inventors: Benedict B. St. Onge, San Marcos, CA (US); David W. Brandmeyer, Newport Beach, CA (US); Richard P. Hooper, Irvine, CA (US)

(73) Assignee: PROMPT CARE INC, NEWPORT BEACH, CA (US)

(21) Appl. No.: 13/262,059

(22) PCT Filed: Mar. 29, 2010
(86) PCT No.: PCT/US10/29095

§ 371 Date: Sep. 29, 2011

Related U.S. Application Data
(60) Provisional application No. 61/164,871, filed on Mar. 30, 2009.
(60) Provisional application No. 61/165,228, filed on Mar. 31, 2009.

(51) Int. Cl. A61L 9/015; B01J 19/08

(52) U.S. Cl. 422/4; 422/186.07; 422/121; 422/119

(57) ABSTRACT

An air treatment system including an ozone generator and a humidifier is disclosed herein. The ozone generator and the humidifier may be configured to communicate with one another. The ozone generator and the humidifier may be configured to communicate with a communications hub. The ozone generator, the humidifier and/or the hub may be configured to communicate with other devices. Methods for using the devices and systems are also disclosed.

### BACKGROUND OF THE INVENTION

1. Field of the Invention

The field pertains to air treatment. More particularly, the field pertains to a system for treating air with ozone and/or hydrogen peroxide.

2. Description of Related Technology

Systems for air treatment, for example, to remove allergens, pathogens, odors, dust, and other such unwanted contaminants are particularly advantageous for buildings which have been damaged by mold or smoke or have been exposed to other airborne contaminants, such as those from cooking, cleaning, and home or industrial use of chemicals.

To treat the air, portable air treatment equipment is typically brought to the building and left to operate for some time. Those operating such systems may or may not stay with the equipment for the entire duration of the treatment.

The air treatment system may introduce oxidizing agents into the air, such as ozone and hydrogen peroxide. Some systems generate ozone with ultra-violet (UV) light. While the agents are helpful for treating the air, if spilled in high concentration, the oxidizing agents can cause damage to various objects, such as furniture and floors. In addition, UV light can be harmful to people, animals, and plants.

### BRIEF DESCRIPTION OF THE DRAWINGS

FIG. 1 is a block diagram of a system used for treating air.

FIG. 2 is a schematic diagram illustrating one embodiment of an ozone generator for use in the system of FIG. 1.

FIGS. 3A and 3B are schematic diagrams, each respectively illustrating embodiments of humidifiers for use in the system of FIG. 1.

FIG. 3C is a schematic diagram of an embodiment of a diffuser for use in the humidifier of FIG. 3B.

FIG. 4 is a schematic diagram of a system used for treating air.

FIGS. 5-7 are photographs of features of one embodiment of an ozone generator.

FIG. 8 is a photograph of one embodiment of a humidifier.

FIGS. 9 and 10 depict a humidifier or ozone generator case.

FIGS. 11A and 11B depict one embodiment of a lock box.

FIG. 12A depicts an embodiment of a mask.

FIG. 12B depicts an embodiment of a filter for the mask of FIG. 12A.

FIG. 13 is a schematic illustration of inventive aspects of a lamp which can be used for the ozone generator of FIG. 2.

### SUMMARY OF THE INVENTION

In one aspect an air treatment system includes an ozone generator, and a humidifier. The ozone generator includes communication circuitry integrated therewith, and the humidifier includes communication circuitry integrated therewith. In addition, the ozone generator and the humidifier are configured to communicate with each other.

In some embodiments, the humidifier is configured to diffuse hydrogen peroxide into the air. In some embodiments, the humidifier is configured to diffuse about 450 ml/hr hydrogen peroxide into the air. In some embodiments, the humidifier is configured to diffuse about 453 ml/hr hydrogen peroxide into the air. In some embodiments, the ozone generator and the humidifier are configured to communicate status information. In some embodiments, the ozone generator and the humidifier are configured to communicate through a wireless connection. In some embodiments, the ozone generator and the humidifier are configured to communicate through a wired connection. In some embodiments, at least one of the ozone generator and the humidifier includes a sensor, and is configured to communicate sensor information. In some embodiments, at least one of the ozone generator and the humidifier is electrically connected to communication circuitry external thereto. In some embodiments, at least one of the ozone generator and the humidifier weighs less than 15 lbs. In some embodiments, the system further includes a communication hub, configured to communicate with at least one of the ozone generator and the humidifier. In some embodiments, the communication hub is further configured to communicate with another communication hub of a separate system. In some embodiments, the communication hub is further configured to communicate with equipment located in a building remote to the system or in a service vehicle. In some embodiments, the communication hub is located within a locked box. In some embodiments, the communications hub communicates via a local cellular network, a wide-area network, or the internet. In some embodiments, the communications hub has a uniform resource locator (URL). In some embodiments, the communications hub has a short range antenna, and a connection for an external extended range antenna. In some embodiments, the communications hub has one or more connections for at least one of a display, a keypad, a pointing device, a printer, and other equipment. In some embodiments, the communications hub is configured to operate on battery power for up to 12 hours without recharge. In some embodiments, the communications hub is portable. In some embodiments, the communications hub weighs less than 15 lbs.

In another aspect an ozone generator device includes a housing, and a lamp within the housing, where the lamp is configured to emit ozone generating light. The device also includes a plurality of walls, including openings therein, where the openings form a plurality of passages for ozone generated by the light to escape from the housing, and the walls prevent the light from escaping from the housing.

In some embodiments, the device further includes a sensor within the housing. In some embodiments, the device weighs less than 15 lbs. In some embodiments, the device further includes a connection for communications circuitry. In some embodiments, the connection includes a wireless connection. In some embodiments, the connection includes a wired connection. In some embodiments, the connection includes a USB or a firewire connection. In some embodiments, the device further includes communications circuitry. In some embodiments, the communications circuitry includes circuitry for wireless communication. In some embodiments, the communications circuitry includes circuitry for wired communication. In some embodiments, the device further includes a sensor, where the communications circuitry is configured to communicate sensor information. In some embodiments, the communications circuitry is configured to communicate status information. In some embodiments, the device further includes a microphone connected to the housing. In some embodiments, the device further includes a speaker connected to the housing. In some embodiments, the communications circuitry is configured to communicate with hardware, a controller, or an individual. In some embodiments, the communications circuitry is configured as a node on a communications network including wired and wireless communication links. In some embodiments, the communications circuitry includes a processor and memory. In some embodiments, the processor and the memory are configured to operate a local operating system and one or more software applications. In some embodiments, one of the software applications is configured to process data so as to generate a result and the communications circuitry is configured to communicate the result. In some embodiments, the communications circuitry is configured to operate with power from at least one of a building, an automobile power, and a self-contained rechargeable battery. In some embodiments, the communications circuitry is uniquely identifiable. In some embodiments, the device is configured to output about 10 grams/hour of ozone. In some embodiments, the light has a first wavelength below about 200 nanometers. In some embodiments, the light has a first wavelength of about 185 nanometers. In some embodiments, the light has a second wavelength of about 254 nanometers. In some embodiments, the openings in the walls are staggered and where a ratio of 185 nm energy to 254 nm energy is about 1. In some embodiments, the lamp includes a mercury vapor lamp. In some embodiments, the lamp is in a controlled air flow environment of about 200 cfm. In some embodiments, the lamp includes extra heavy duty tungsten conductors. In some embodiments, the lamp includes an 800 ma, 96 watt mercury lamp. In some embodiments, the lamp includes a quartz u-tube about 17 inches in length. In some embodiments, the walls include aluminum. In some embodiments, the walls include a material which absorbs the light from the lamp. In some embodiments, the walls include a coating which absorbs the light from the lamp. In some embodiments, the lamp is mounted to the housing with a fluorosilicone isomer.

In another aspect a method of treating air includes providing an ozone generator with communication circuitry, providing a humidifier with communication circuitry, sending a first signal from the ozone generator to the humidifier, and sending a second signal from the humidifier to the ozone generator. In some embodiments, the first signal includes status information. In some embodiments, the second signal includes status information. In some embodiments, the method further includes diffusing hydrogen peroxide into the air with the humidifier. In some embodiments, the method further includes diffusing about 450 ml/hr hydrogen peroxide into the air with the humidifier. In some embodiments, the method further includes diffusing about 453 ml/hr hydrogen peroxide into the air with the humidifier. In some embodiments, the method further includes outputting about 10 grams/hour of ozone with the ozone generator. In some embodiments, sending the first signal includes sending the first signal through a wireless channel. In some embodiments, sending the first signal includes sending the first signal through a wired channel. In some embodiments, at least one of the ozone generator and the humidifier includes a sensor, and at least one of the first and second signals includes sensor information. In some embodiments, the method further includes sending a third signal from at least one of the humidifier and the ozone generator to a communications hub. In some embodiments, the method further includes sending a fourth signal from the communication hub a second communication hub of a separate system. In some embodiments, the method further includes sending a fifth signal from the communication hub to equipment located in a building remote to the system or in a service vehicle. In some embodiments, the method further includes emitting ozone generating light with a lamp within the ozone generator. In some embodiments, the method further includes sending a sixth signal from one of the humidifier and the ozone generator to a controller or to an individual. In some embodiments, at least one of the humidifier and the ozone generator includes a processor and a memory, and the method further includes the processor and the memory operating a local operating system and one or more software applications. In some embodiments, one of the software applications is configured to process data so as to generate a result and one of the first and the second signals includes the result. In some embodiments, the method further includes operating the humidifier and the ozone generator with power from at least one of a building, an automobile power, and a self-contained rechargeable battery.

In another aspect a method of manufacturing an ozone generator device includes providing a housing, and mounting a lamp within the housing, where the lamp is configured to emit ozone generating light. The method also includes connecting a plurality of walls to the interior of the housing, where the walls include openings therein, and where the openings are configured to allow ozone generated by the light to escape from the housing and to prevent the light from escaping from the housing.

In another aspect, a humidifier includes a housing enclosing a first chamber and a second chamber, the first chamber holding a hydrogen peroxide solution, and the second chamber coupled to an ultra sonic mechanism configured to vaporize hydrogen peroxide in the second chamber. The second chamber is in fluid communication with the first chamber and the second chamber is in fluid communication with ambient atmosphere such that vaporized hydrogen peroxide can enter the atmosphere.

In some embodiments, the hydrogen peroxide solution is isolated from the outside when the humidifier is upended, inverted or left on its side.

In another aspect, a humidifier includes an air inlet, an air outlet, an evaporator, and a fan configured to force air from the air inlet through the evaporator and out of the humidifier through the air outlet. The humidifier also includes a chamber configured to hold a solution, a pump, and a fluid circulation path including the pump, the chamber, and the evaporator, where at least a portion of the solution exits the evaporator in the air forced through the evaporator.

In some embodiments, the evaporator includes a diffusion layer, configured to allow the air to flow therethrough and to allow the solution to flow therethrough. where at least a portion of the solution exits the diffusion layer in the air, and first and second air permeable layers on opposite sides of the diffusion layer. In some embodiments, the evaporator further includes first second screens, where the first air permeable layer is compressed between the first screen and the diffusion layer, and the second air permeable layer is compressed between the second screen and the diffusion layer.

In another aspect, an ozone generator device includes a housing, including an air inlet, and an air outlet, and a lamp within the housing, where the lamp is configured to emit ozone generating light. The lamp includes a glass enclosing a gas, one or more pins, configured to electrically connect to a power source, a filament within the glass, and first and second electrodes extending through the glass and electrically connected to the pin. The filament is electrically and mechanically connected to the first and second electrodes. The ozone generator also includes a fan configured to force air from the air inlet out of the device through the air outlet, where the air exiting the device includes ozone.

In some embodiments, the filament is medium gauge. In some embodiments, the filament includes first and second ends and the filament is connected to the first and second electrodes at the first and second ends, respectively.

In another aspect, a method of introducing a solution into air includes providing a humidifier including an air inlet and an air outlet, pumping a solution from a storage chamber to an evaporator, collecting the solution from the evaporator in the storage chamber, and propagating air from the air inlet through the evaporator to the outlet, where a portion of the solution enters the air in the evaporator and exits the humidifier with the air through the air outlet.

In some embodiments, the method further includes forcing air to flow through a diffusion layer in the evaporator, and providing solution to flow through the diffusion layer, where a portion of the solution exits the diffusion layer with the air. In some embodiments, pumping the solution includes pumping the solution to a top of the evaporator, where the evaporator is vertically oriented. In some embodiments, propagating the air includes forcing the air with a fan.

### DETAILED DESCRIPTION OF THE PREFERRED EMBODIMENT

The following description and examples illustrate some exemplary embodiments of certain inventive aspects. Those of skill in the art will recognize that there are numerous variations and modifications that are encompassed by the scope of the disclosure and the appended claims. Accordingly, the description of any particular exemplary embodiment should not be deemed to limit the scope of the present description.

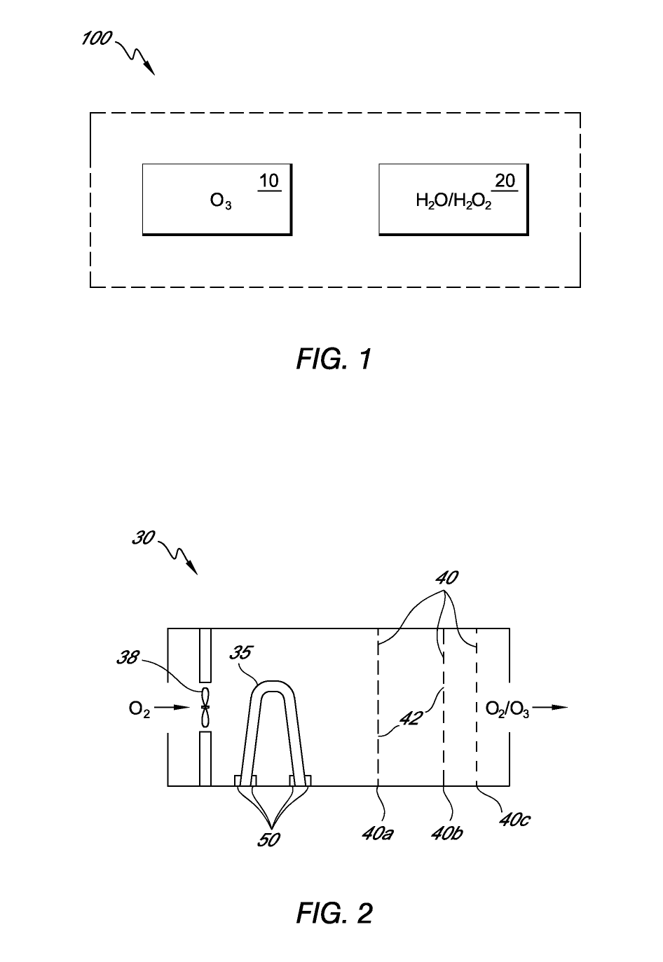

FIG. 1 is a block diagram of a system 100 used for treating air. The system 100 includes an ozone generator 10 and a humidifier 20. In some embodiments the humidifier 20 is configured to diffuse hydrogen peroxide into the air. The ozone generator 10 and the humidifier 20 are configured to cooperatively operate to treat air. The system 100 may be configured to treat the air according to methods described in U.S. Pat. No. 7,407,624, issued Aug. 4, 2008, which disclosure is incorporated herein by reference in its entirety. In some embodiments the ozone generator 10 and humidifier 20 have switches, hour meters, and/or an additional plug for other equipment, such as other ozone generators, humidifiers and fans.

In some embodiments the ozone generator 10 and humidifier 20 have communications circuitry capable of communicating with each other and/or with other equipment. One such embodiment is discussed further below. In some embodiments the communications circuitry is integrated with the ozone generator 10 and the humidifier 20. In some embodiments the communications circuitry is included in a separate piece of equipment and communicates with at least one of the ozone generator 10 and the humidifier 20 through a wire, such as a USB connection, firewire or other wired standard computer interface connections. In some embodiments the communications circuitry communicates wirelessly with at least one of the ozone generator 10 and the humidifier 20. In some embodiments the ozone generator 10 and the humidifier 20 may be configured to communicate status information, sensor information and/or verification information with each other and/or with other equipment. In some embodiments the ozone generator 10 and the humidifier 20 communicate with one another. In some embodiments the ozone generator 10 and the humidifier 20 additionally or alternatively communicate with other equipment. In some embodiments one or both of the ozone generator 10 and the humidifier 20 include environmental, video and/or audio sensors. In some embodiments environmental, video and/or audio sensors, and/or the communication capabilities of the ozone generator 10 or the humidifier 20 may be used to communicate with hardware, a controller, or an individual. The hardware may be, for example, part of a network or a stand alone computer. The controller may be, for example, a local or a remote device.

In some embodiments the communication circuitry serves as a node on a communications network comprising wired and wireless communication links. Each communication circuit may include, for example, a processor with local memory capable of operating on household current, generator power and/or a self-contained rechargeable battery. The communication circuit may also have network adapters, antennas and/or connectors to conform to the communications network protocols. The processor may have a local operating system and have provisions for custom software applications. Each communications circuit may be uniquely identifiable. In addition, the communications circuits may interface with either the ozone generator or the humidifier equipment and receive or sense the operating state of the associated equipment (for example, powered-on or powered-off). Local processing of data by the communications circuitry may be performed. This may minimize the amount of data transmitted from or received by the communications circuitry.

The ozone generator 10 and the humidifier 20 each may be designed to weigh less than 15 lbs. In some embodiments the ozone generator 10 and/or the humidifier 20 may be housed in a case having rounded corners and formed of a deformable material to minimize worksite area damage, survive transit environment and withstand the continual exposure to high concentrations of oxidants. In some embodiments the humidifier 20 diffuses about 450 ml/hr hydrogen peroxide into the air. For example, the humidifier 20 may diffuse about 453 ml/hr hydrogen peroxide into the air using a storage containing a 3% hydrogen peroxide solution. In some embodiments ultrasonic energy is used to create micro particles which are then diffused into a 200 cfm air stream.

The ozone generator 10 outputs up to and including about 20 grams/hour of ozone using custom lamp technology. For example, to generate ozone with a lamp, a gas within the lamp is ionized by a current. The excited gas emits an electromagnetic energy having a wavelength below about 200 nanometers. The electromagnetic energy transforms O₂ into O₃. In some embodiments a mercury vapor lamp may be used. The ionized mercury vapor has two emission peaks, one at about 254 nm and one at about 185 nm. In some germicidal UV lamps, only the 254 nm peak is detectable unless the outer lamp material is made of fused quartz. Another aspect is a lamp may produce energy emission ratios of about 1 unit of 254 mm energy for every 1 /4 unit of 185 nm energy. However, the 254 nm energy and the 185 nm energy do not cooperatively generate O₃. The 185 nm emissions transform 3 O₂s into 2 O₃s, but the 254 nm emissions destroy O₃ by transforming 2 O₃s into 3 O₂. Accordingly, it is desirable to adjust the generation environment such that more O₃ is generated and less O₃ is destroyed. In some embodiments the ozone generator 10 also incorporates thermal and current fuses to cut power in the event of equipment failure.

One feature for producing more O₃ increases the ratio of 185 nm emissions to 254 nm emissions. This can be accomplished, for example, by adjusting the temperature of the ionized gas. Lamp current for a mercury vapor lamp can be selected to manipulate a ratio of emissions. As the ionized gas temperature rises, the emission level of the 254 nm energy decreases while the 185 nm emission remains substantially constant. Accordingly, a higher temperature results in an increased ratio of 185 nm emissions to 254 nm emissions. In some embodiments the ratio of 185 nm energy to 254 nm energy is about 1. This results in higher levels of ozone production when compared to conventional methods. In some embodiments, the temperature is preferably between about 100° F. and about 200° F.

In some embodiments the lamp is placed in a controlled air flow environment in order to prevent lamp breakage. For example, about 200 cfm of air flow over the lamp may be used to prevent overheating and breakage. Additionally or alternatively, because the increased temperature leads to increased pressure in the lamps, extra heavy duty tungsten conductors may be used with the end filaments. The heavy duty conductors may be configured to withstand the extra power and mechanical shock. In some embodiments 800 ma (96 watts) mercury lamps with extra heavy duty tungsten conductors are used. In some embodiments the tungsten filaments are formed with an oversized gauge when compared to conventional lamps to accommodate the additional current. This also gives much better resistance to mechanical shock. In addition, the filaments may be designed for 4 wire ballasts, but the lamps themselves may be 2 wire. The additional second support secures the filament from both ends similar to a conventional light bulb design, which further increases the shock and vibration rating needed for rough transport portable equipment. In some embodiments the lamps are custom made for the ozone generator 10. In some embodiments the lamp envelopes are made of heavy wall quartz u-tubes of about 17 inches in length.

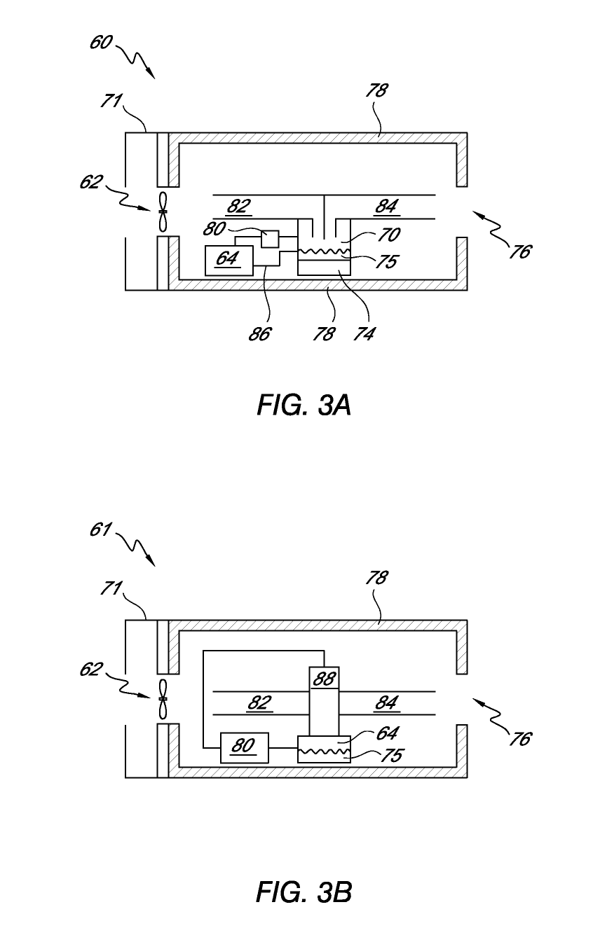

Another aspect is a second feature for producing more O₃ reduces the amount of time the O₃ is exposed to 254 nm emissions. FIG. 2 is a schematic diagram illustrating an ozone generator 30 which can be used in system 100 of FIG. 1. The ozone generator 30 has features for reducing the amount of time the generated O₃ is exposed to 254 nm emissions. The ozone generator 30 may include a fan 38, a lamp 35 and walls 40. The walls 40 may be opaque to the lamp light and have openings 42, which allow the generated O₃ to exit. Because of the Venturi effect, the pressure at the openings is lower than the pressure at the fan 38. Once the O₃ passes through the openings 42 of the first wall 40a, the O₃ is shaded from the O₃ destroying 254 nm emissions from the lamp by the non-opening portions of the first wall 40a. Likewise, once the O₃ passes through the openings 42 of the second wall 40b, the O₃ is shaded by the non-opening portions of the first and second walls 40a and 40b from the 254 nm emissions. In some embodiments once the O₃ has passed the last wall 40c, the O₃ is completely shaded from the lamp 35 by the walls 40. In some embodiments more than three walls are used. In some embodiments fewer than three walls are used. In some embodiments multiple walls having slits of differing orientation are used. Additional beneficial aspects of this arrangement can include that the light from the lamp, which light can be harmful to people, does not escape the ozone generator 30 due to the staggered configuration of openings 42 in the first wall 40a, the second wall 40b and the third wall 40c. In some embodiments the walls 40 may include aluminum. In some embodiments the walls 40 may include a material which absorbs the light from the lamp. In some embodiments the walls 40 may include an oxide coating.

To protect the lamp 35 from mechanical shock, the lamp 35 may be mounted to the ozone generator 30 with a fluorosilicone isomer 50. The mount 50 may be effective in absorbing mechanical shock and may be resistant to the corrosive effects of the environment due to any of the ozone, the light and/or the hydrogen peroxide.

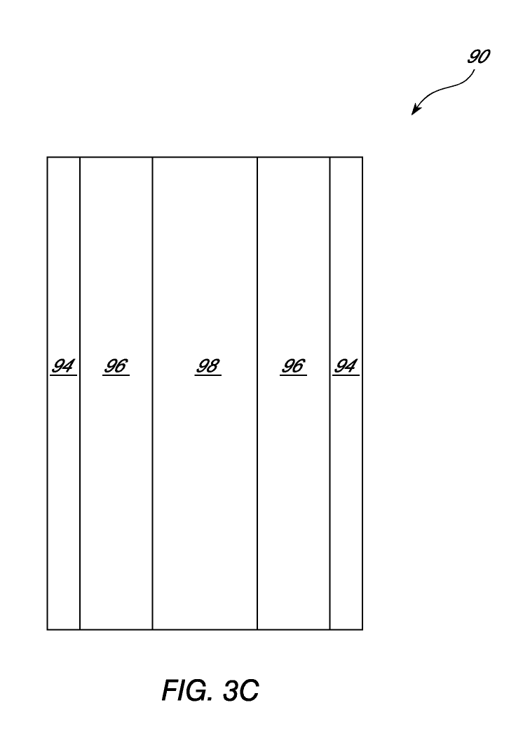

FIG. 3A is a schematic diagram illustrating a humidifier 60 which can be used in system 100. The humidifier 60 includes a case 71, a fan 62, air paths 82 and 84, a primary storage chamber 64, an ultrasonic vaporizer chamber 70 containing solution 75, an ultrasound source 74, a pump 80, and absorption media 78. Another aspect is a water and hydrogen peroxide solution 75 is stored in the primary storage chamber 64.

The fan 62 forces air through the case 71 which exits through the outlet 76. At least a portion of the forced air travels through air path 82 into the ultrasonic vaporizer chamber 70, where it mixes with diffused hydrogen peroxide, before continuing through air path 84, and out the outlet 76.

The water and hydrogen peroxide solution is provided to the storage chamber 64 through a filler tube (not shown), while gas escapes through a vent (not shown). The solution is introduced to the ultrasonic vaporizer chamber 70 with pump 80. In some embodiments, the depth of the solution within the vaporizer chamber 70 is maintained substantially constant. For example, the depth may be maintained at about 1 inch, about 1.5 inches, or about 2 inches. In some embodiments, the overflow path 86 maintains the level substantially constant. The overflow path 86 may be gravity driven and in some embodiments, includes multiple paths. In some embodiments, at least one overflow path 86 is smaller than another overflow path 86. For example, the smallest of the overflow paths 86 may be sized so that it is substantially filled with solution returning from the vaporizer chamber to the storage chamber 64. In some embodiments, the overflow path 86 comprises paths which join before connecting with the storage chamber 64. For example, overflow path 86 may include two tubes which are each connected to the vaporizer chamber 70, where one tube is ¼ inch tubing and the other is 1/16 inch tubing. The 1/16 inch tube may join the ¼ inch tube with a “T” junction, from which the overflow path 86 continues with a ¼ inch tube from the “T” junction to the storage chamber 64.

Within the chamber 70, the solution is vaporized by the ultrasound source 74. The ultrasound source 74, may be, for example, an about 20-40 KHz ultrasound device. The ultrasonic energy from the ultrasound source 74 causes a portion of the solution to vaporize. The air flow through air paths 82 and 84 carries the vaporized solution out of humidifier 60 through outlet 76.

Spillage of the hydrogen peroxide solution can cause damage to other items, such as carpets and furniture. The humidifier 60 may be configured to effectively inhibit spillage of the hydrogen peroxide solution when the portable equipment is upended, inverted or left on its side. To accomplish this, the storage chamber 64, the vaporizer chamber 70, and one or more additional storage chambers are in fluid communication and include sufficient capacity that when the portable equipment is upended, inverted or left on its side the solution does not flow into the air paths 82 and 84. For example, for 1 gallon of solution and additional at least 1 gallon of chamber capacity may be used. In some embodiments the primary storage chamber 64 holds approximately 1 gallon of the hydrogen peroxide solution, thus limiting the possible spillage therefrom to approximately 1 gallon. In some embodiments, the vaporizer chamber 70 is sized and shaped so that if the humidifier 60 is upended, inverted or left on its side, the level of the solution is isolated from the entrance to the air paths 82 and 84 and is therefore isolated from the outside. In some embodiments, a third chamber (not shown) is in fluid communication with the storage chamber 64 and the vaporizer chamber 70, such that if the humidifier 60 is upended, inverted or left on its side, the level of the solution is isolated from the entrance to the air paths 82 and 84, and is therefore isolated from the outside. In some embodiments, there is a gap between each of the air paths 82 and 84 and the case 71, so that even if solution escapes from the vaporizer chamber 70, the solution remains within the case 71, and is adsorbed by adsorption media 78 within the case 71. In some embodiments, adsorption media 78 is configured to absorb spillage and will renew itself during transit or subsequent operation by slowly releasing adsorbed solution.

FIG. 3B is a schematic diagram illustrating a humidifier 61 which can be used, for example, in system 100. The humidifier 61 includes a case 71, a fan 62, air paths 82 and 84, an evaporator 88, a primary storage chamber 64 containing solution 75, a pump 80, and absorption media 78. Another aspect is a water and hydrogen peroxide solution 75 is stored in the primary storage chamber 64.

The fan 62 forces air through the case 71 which exits through the outlet 76. At least a portion of the forced air travels through air path 82 to the evaporator 88, where it mixes with hydrogen peroxide. The air continues through air path 84 and out the outlet 76.

Solution 75 is pumped by pump 80 from the storage chamber 64 to evaporator 88. The solution 75 flows through evaporator 88, where a portion of the solution 75 evaporates or diffuses into the air flowing through the evaporator 88. In some embodiments, the solution 75 is pumped through the evaporator 75 by the pump 80. In some embodiments, the solution flows through evaporator 75 because of gravitational pull. The solution 75 flows from the evaporator 75 to the storage chamber 64.

FIG. 3C is a schematic diagram illustrating an evaporator 90, which is an embodiment of an evaporator, which may be used, for example, in the humidifier 61 of FIG. 3B. Evaporator 90 includes screens 94, air permeable layers 96, and diffusion layer 98. In this embodiment, the air flows from one vertical side of the evaporator 90 to another. Air flows into the evaporator through a first screen, and continues through a first air permeable layer 96, through the diffusion layer 98, through a second air permeable layer 96, and out of the evaporator 90 through a second screen 94.

Diffusion layer 98 is configured to allow the solution 75 to flow therethrough. In this embodiment, the solution 75 flows from top to bottom due to gravitational pull. As discussed above, the diffusion layer 98 is configured to allow air to flow therethrough. As the air passes through the diffusion layer 98, it mixes with a portion of the solution 75 to achieve or maintain a desired concentration of the solution 75 in the air. The diffusion layer 98 is substantially inert to the solution 75. The diffusion layer 98 is absorbent so that it may function to wick up the solution. In some embodiments, the diffusion layer 98 may include, for example, at least one of cotton, fiberglass, and polyethylene fabric. In some embodiments, the diffusion layer 98 is formed about 1/16 inch thick.

In this embodiment, the diffusion layer 98 is sandwiched by air permeable layers 96. The air permeable layers 96 are configured to allow air to flow therethrough both to and from the diffusion layer 98. In addition, the air permeable layers 96 may provide mechanical support to the diffusion layer 98 as a result of the air permeable layers 96, which may be mechanically compressed between one of the screens 94 and the diffusion layer 98. Some of the mechanical support for the diffusion layer 98 coming from outside the diffusion layer 98 may allow for optimization of solution flow and air flow properties of the diffusion layer 98 while easing mechanical structure constraints. In some embodiments, the air permeable layers 96 are formed about ½ inch thick. In some embodiments, the permeable layers 96 comprise at least one of polypropylene and polyethylene fiber material having fibers with diameter about 0.005 inches.

The screens 94 allow air to flow to and from the air permeable layers 96 and provide mechanical support for the air permeable layers 96. In some embodiments, the screen 94 is a ½ inch by ½ inch grid. In some embodiments, the screens 94 include metal.

In some embodiments, the evaporator 90 has dimensions of about 6 inches by about 7 inches by about 1¼ inches. The amount of solution 75 which is provided to the evaporator 90 is determined so that solution 75 is available for gaseous diffusion into the air, and so that air flows through the evaporator without forcing large amounts of liquid solution, or in some embodiments, any liquid solution out of the evaporator 90.

In some embodiments, the evaporator 90 includes multiple diffusion layers 98, each of which is sandwiched by air permeable layers 96.

In some embodiments high pressure misting heads targeted at a drain back system allow the proper sized droplets to be picked up and carried away with the air stream. In some embodiments the droplets generally range in size from about 1 μm to about 10 μm with and average of about 5 μm for an ultra sonic method, and range from about 10 μm to about 1000 μm for a spray method. For the spray method the water surface area may be significant. One advantage of using the misting heads may include weight savings, which are possible because the power conditioning requirements to run low voltage high power ultrasonics can be eliminated. Another advantage of using the misting heads is that the maximum humidity levels in the room can be automatically regulated because the system meters less in humid conditions and more in dry conditions, such as at start up. In some embodiments regulation is accomplished automatically by vapor deficit calculations from 100%, such that dryer air results in a greater evaporation rate.

In some embodiments the ozone generator 10 and/or humidifier 20 of FIG. 1 also include one or more condition monitoring sensors. The sensors may be used to monitor, for example, any of ozone concentrations, peroxide concentrations, humidity, temperature and/or air flow. In some embodiments sensors monitoring a status of the ozone generator 10 or humidifier 20 can be used. For example a sensor may monitor the output level of the ozone generator 10 or humidifier 20, and/or whether the ozone generator 10 or humidifier 20 is operating within specification. In some embodiments the sensors include one or more cameras configured to generate still images or video images. In some embodiments the sensors include one or more microphones.

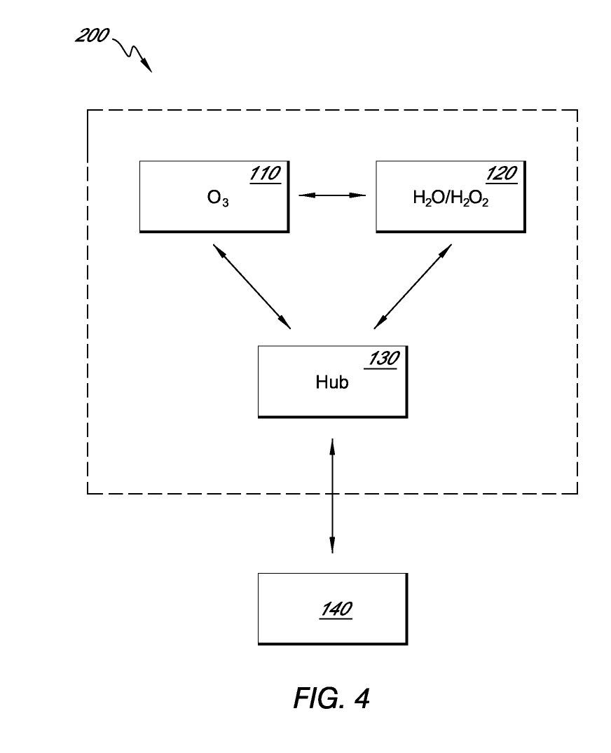

FIG. 4 depict a schematic diagram of system 200 used for treating air. The system 200 includes an ozone generator 110, a humidifier 120, and a communication hub 130. The ozone generator 110 and the humidifier 120 are configured to communicate with communication hub 130 and may, for example, be similar to ozone generators and humidifiers discussed above. The system 200 is also configured to communicate with other equipment 140. The other equipment 140 may be located at, for example, a central office or a field technician's vehicle. Thus, in some embodiments the ozone generator 110, the humidifier 120 and/or the communication hub 130 can communicate with the other equipment 140.

The communications hub 130 is capable of communicating with other hubs and/or communications circuits, and/or with other equipment 140. In some embodiments the communications hub 130 is located within a lockable box which is securely located on the outside of a building being serviced. In some embodiments the lockable box has an attachment member configured to connect the box to a standard door knob. In some embodiments the communications hub 130 has similar capabilities and features as the communications circuits described above. In addition, the communications hub 130 serves as an interface between local communications circuits operating at a service site and the other equipment 140. In some embodiments the communications hub 130 communicates via a local cellular or other wide-area network. In some embodiments the communications hub 130 communicates via the Internet. In some embodiments the communications hub 130 has a uniform resource locator (URL). The communications hub 130 may be located on a service vehicle, such as a truck parked within communications range of the communication circuits at the service site. In some embodiments in addition to a short range antenna, the communications hub 130 has a connection for an external extended range antenna. The communications hub 130 may also have connections for a display, a keypad, a pointing device, a printer, and/or other equipment. In some embodiments the communications hub 130 is capable of operating on battery power for up to 12 hours without recharge or connection to external power sources. In some embodiments the communications hub 130 is portable and weighs less than 15 lbs.

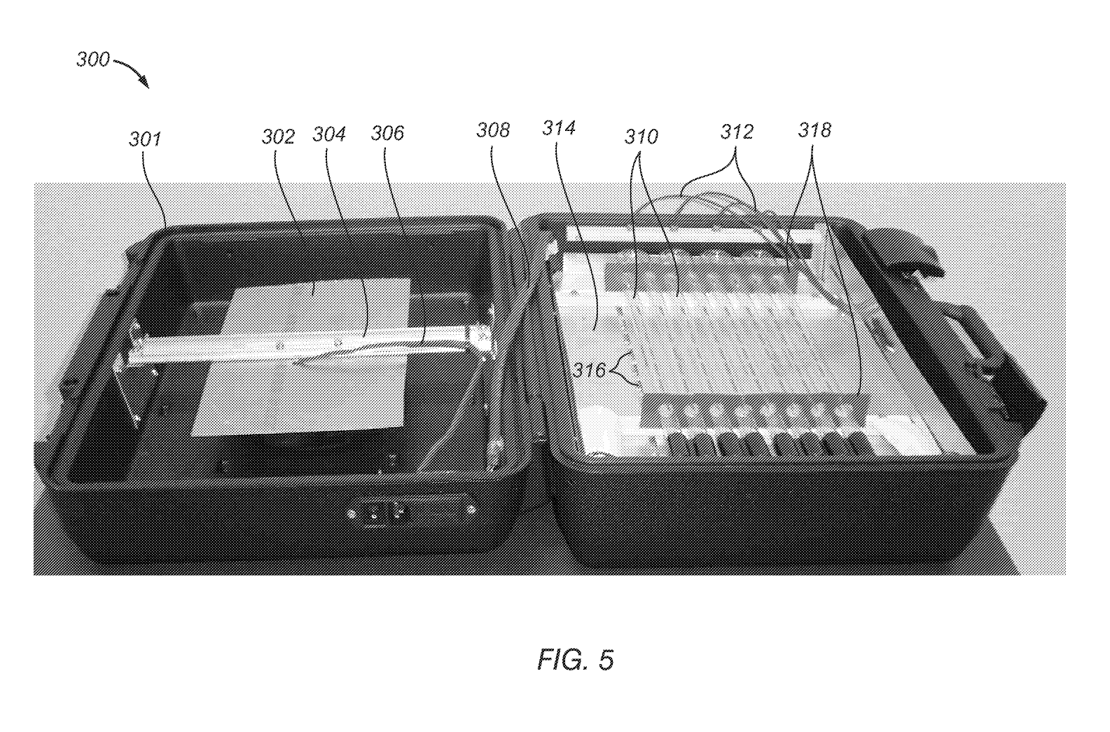

FIG. 5 is a photograph of an embodiment of an ozone generator 300. In this embodiment, within case 301, a fan (not shown) behind plate 302 is supported by support member 304. Cables 306 and 308 provide electrical power to the fan and the lamps 310, respectively. Cables 306 and 308 may be covered by an insulation and/or protective material configured to withstand the corrosive environment of the ozone generator 300. In some embodiments the insulation and/or protective material on cables 306 and 308 can withstand up to and including 10,000 hours of operation. Lamps 310 are shown as held in place by lamp fixture 318. Beneath the lamps 310 is shown wall 314 having openings 316.

During operation the case 301 is closed. The fan draws ambient air into the case 301. The lamps 310 operate to generate electromagnetic energy, which induces ozone generation. Because of the air flow caused by the fan, the ozone is forced through the openings 316 and out of the case. In addition, each of a plurality of optical conductors 312 carry light safely visible from outside the case, which indicate that the lamps 310 are operating.

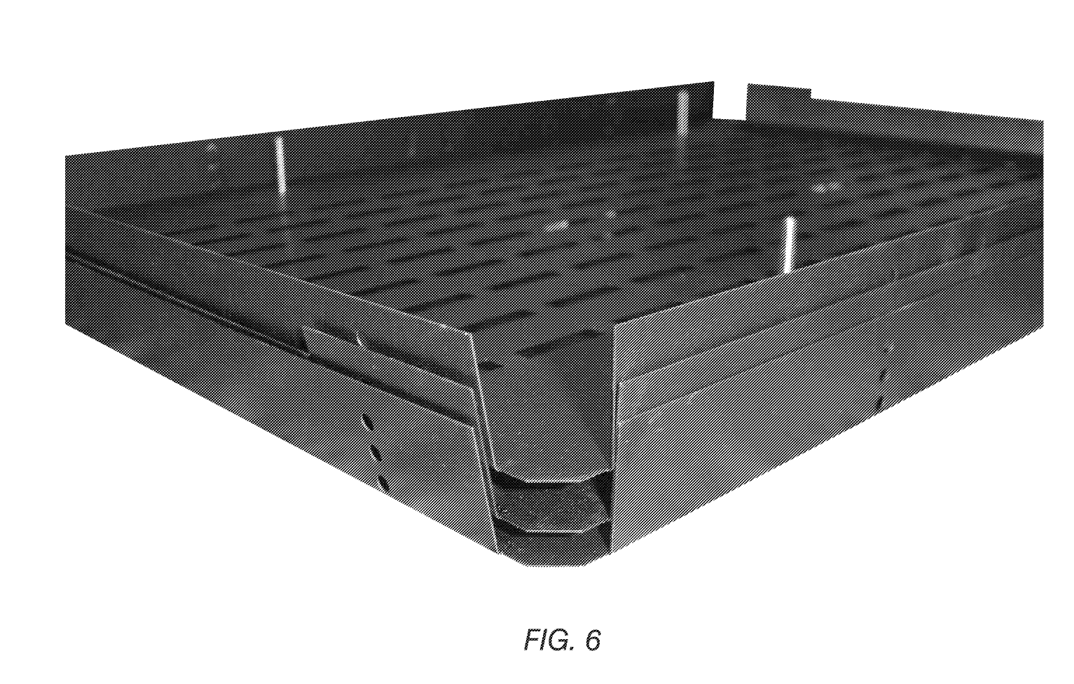

FIG. 6 is a photograph of three walls, such as wall 314 of FIG. 5 and walls 40 of FIG. 2. In this embodiment openings in each wall are rectangular slots and the walls and coating materials are configured to absorb light from a lamp, such as the lamps 310 of FIG. 5.

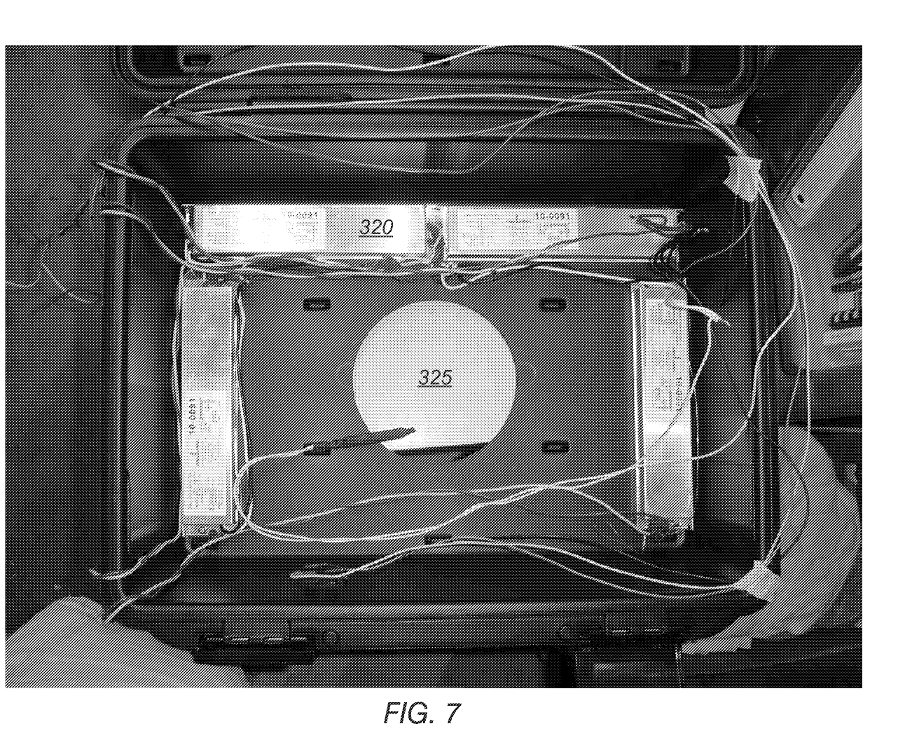

FIG. 7 is a photograph of case 301 of ozone generator 300 illustrated in FIG. 5, where the lamps 310 and the walls have not yet been installed. The case 301 has power sources 320 for the lamps and an opening 325 through which the generated ozone exits the case 301.

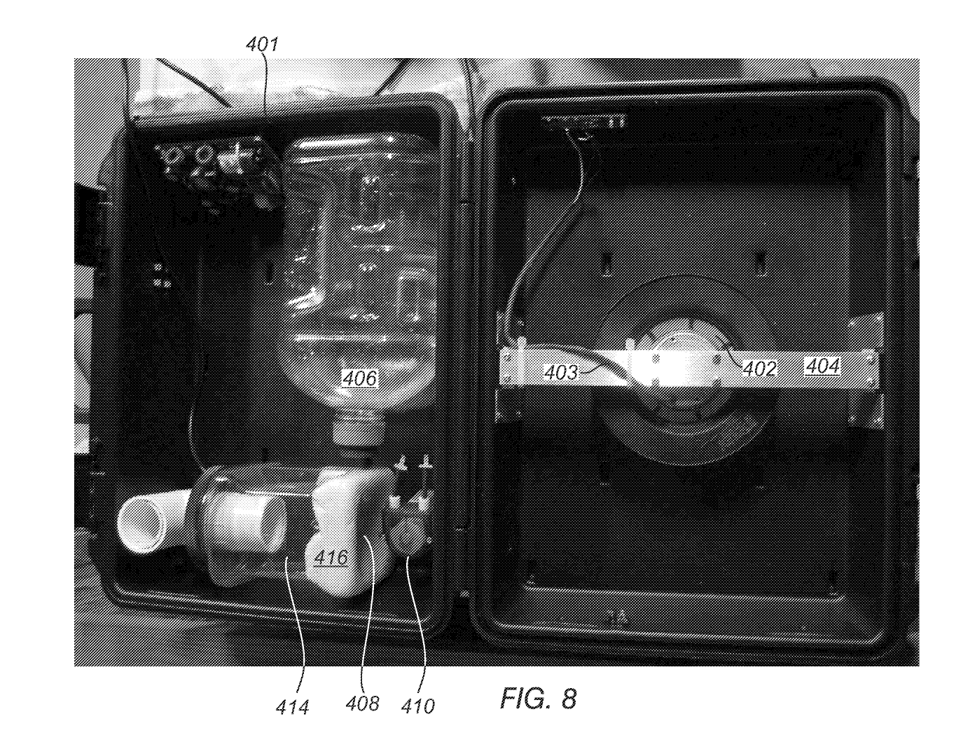

FIG. 8 is a photograph of one embodiment of a humidifier 400. In this embodiment within case 401 is a fan 402 supported by support member 404. Cable 403 provides power to the fan 402 and is configured to withstand the corrosive environment of the humidifier 400. Primary storage chamber 406 holds a hydrogen peroxide solution. The hydrogen peroxide solution may be provided to an ultrasonic vaporizer chamber 414, where it is vaporized by the ultrasound source at location 408 surrounded by foam 416 within the chamber 414. In this embodiment, the pump 410 is located adjacent to the vaporizer chamber 414.

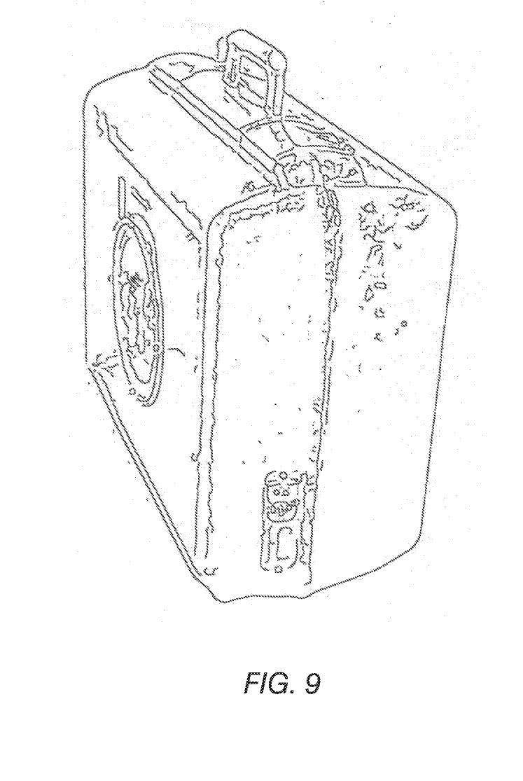

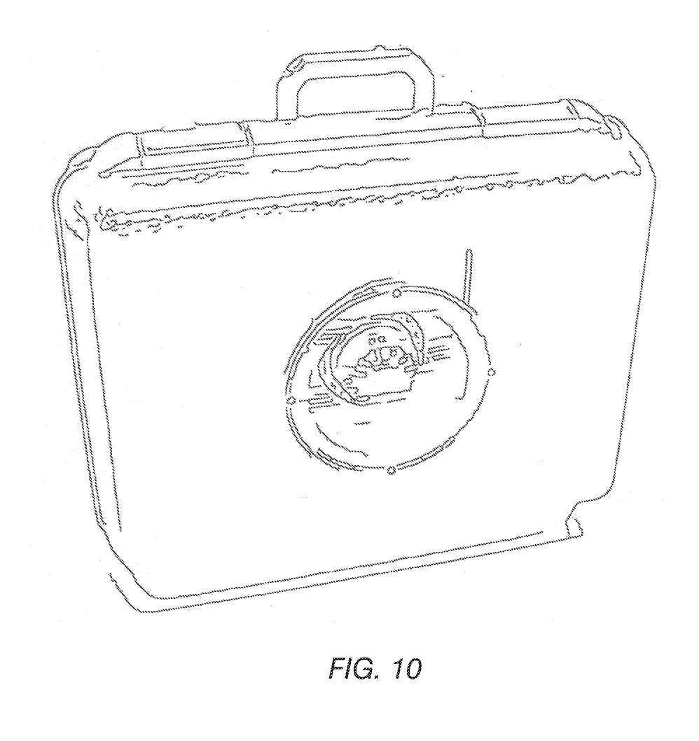

FIGS. 9 and 10 depict a case which can be used for a humidifier and/or an ozone generator case. In some systems the cases used for the humidifier and the ozone generator are substantially identical. The case may be deformable, and may be formed of a material comprising at least one of a plastic, a polymer, polyethylene and polypropylene. In some embodiments, the case has an inner and an outer layer of material with a gap therebetween. In some embodiments, the outer layer is thinner and more flexible than the inner layer.

The cases allow for axial air flow into and out of the cases. In some embodiments, the cases are sized so as to correspond to a standard racking system in a transport vehicle.

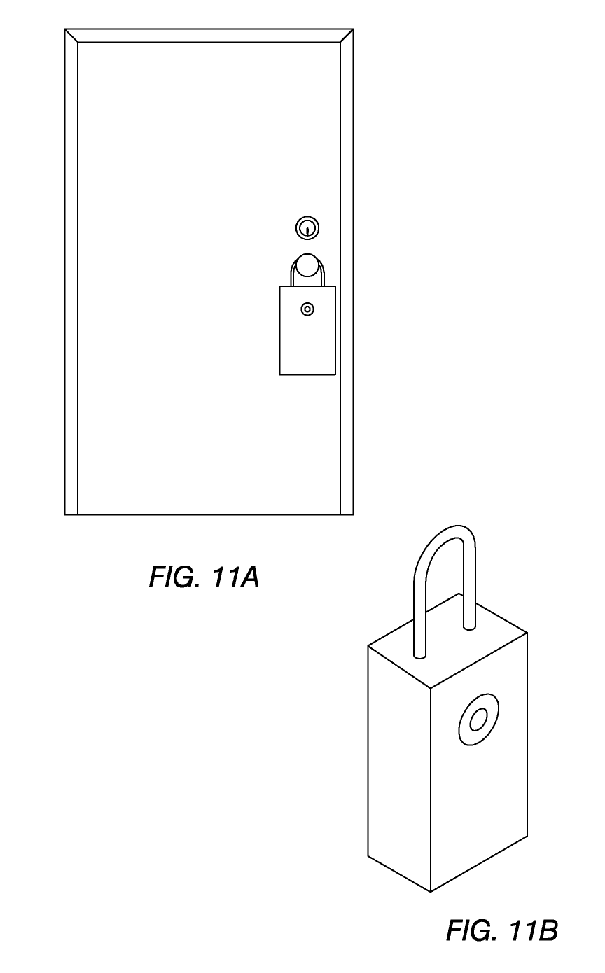

FIGS. 11A and 11B depict an embodiment of a lock box for the communication hub. As shown, this embodiment is configured to be stored on a door knob and may include a combination lock and electronics (contained within the lock box). As discussed above with regard to the system FIG. 4, the lock box illustrated in FIGS. 11A and 11B may be in communication with one or more of a humidifier and an ozone generator. The lock box may further be in communication with other local or remote equipment. The lock box may also be in communication with a network via a wired or wireless connection.

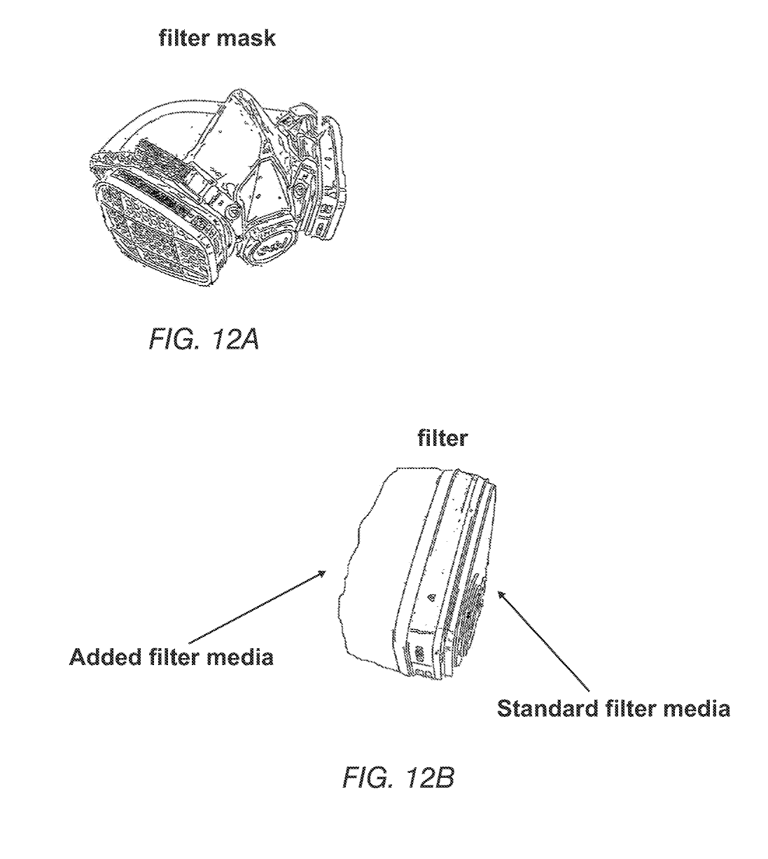

An operator of an air treatment system may be susceptible to lung damage from breathing the ozone and or the hydrogen peroxide used in the process. As a replacement for the current tank fed breathing apparatus, a cartridge based filter mask can be used. FIG. 12A is a drawing of an embodiment of such a mask. As shown, the filter mask may be a face and mouth covering device with one or more cartridges. The filter mask includes an ozone and/or hydrogen peroxide cartridge (such as that shown in FIG. 12B) configured to transform ozone and/or hydrogen peroxide into water vapor and oxygen.

The ozone and/or hydrogen peroxide cartridge illustrated in FIG. 12B may be used in addition to standard mask air treatment, such as large particle filtration, carbon media filtration, and HEPA filtration. The ozone and/or hydrogen peroxide cartridge includes, for example, an ozone destruct media, such as a layer of metal oxide based ozone destruct media. Other ozone destruct media may alternatively or additionally be used. Another aspect is a beneficial result is that the carbon monoxide created when carbon is used as a destruct media may be significantly reduced or eliminated.

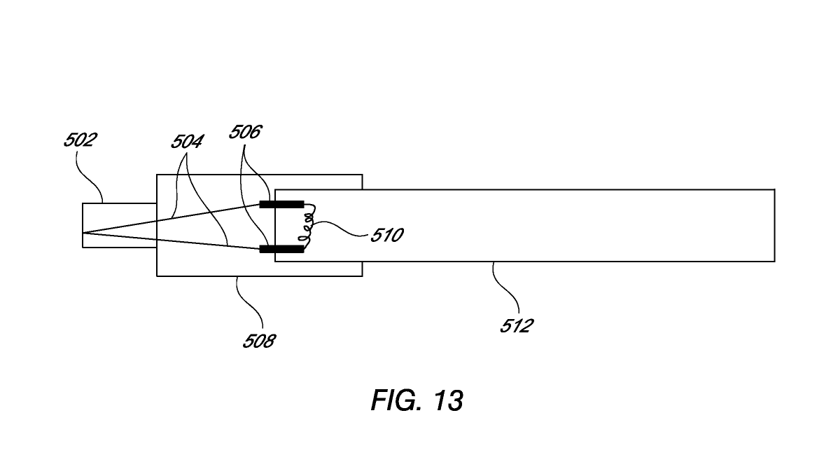

FIG. 13 is a schematic illustration of inventive aspects of a lamp which can be used, for example, with the ozone generator of FIG. 2. Lamp 500 includes a pin 502, wires 504, electrodes 506, filament 510, base 508, and glass 512. In some embodiments, the glass 512 comprises quartz.

The pin 502 electrically connects the lamp 500 to a power source. Wires 504 conduct current to and from the electrodes 506, which extend through the glass 512 and electrically and mechanically contact the filament 510. The base 508 provides mechanical structure and connects the pin 502 and the glass 512.

In operation, current flows through the pin 502, the wires 504, and the electrodes 506 to the filament 510. From the filament 510, the current arcs through gas within the glass 512 to another end (not shown) of the lamp 500, where there is present another filament, additional electrodes and wires, and another pin (not shown).

In conventional lamps, there is only one electrode connecting to the filament. Accordingly, conventional lamps suffer from poor reliability. The connection between the filament and the electrode is susceptible to breakage as a result of mechanical shock.

Lamp 500 includes two electrodes 506, which are each connected to filament 510. Because the filament 510 is connected at both ends, it receives mechanical support at both ends, and is, therefore, much more resistant to breakage as a result of mechanical shock. In addition, if the filament 510 were to break at one end or somewhere in the middle, the lamp 500 would continue to function, because the filament or filament portions would still be connected to the electrodes 506 at the end opposite the point of breakage.

The filament 510 can be of any gauge. In some embodiments, filament 510 is medium gauge. In some embodiments, filament 510 is heavy gauge.

In some embodiments, the lamp 500 uses light weight and/or high frequency electronic ballasts to limit current.

### Abated Substances

The systems of preferred embodiments can be employed to abate any of the substances discussed herein, but are particularly preferred for abatement of pathogens, molds, allergens, and Volatile Organic Compounds (VOCs).

Pathogens that can be controlled include, but are not limited to Anthrax (Bacillus anthracis); Botulism (Clostridium botulinum toxin); Brucella species (brucellosis); Brucellosis (Brucella species); Burkholderia mallei (glanders); Burkholderia pseudomallei (melioidosis); Chlamydia psittaci (psittacosis); Cholera (Vibrio cholerae); Clostridium botulinum toxin (botulism); Clostridium perfringens (Epsilon toxin); Coxiella burnetii (Q fever); E. coli O157:H7 (Escherichia coli); emerging infectious diseases such as Nipah virus and hantavirus; Norwalk virus; Severe Acute Respiratory Syndrome (SARS); Acquired Immune Deficiency Syndrome (AIDS) virus; Human Immunodeficiency Virus (HIV); Epsilon toxin of Clostridium perfringens; Food safety threats (e.g., Salmonella species, Escherichia coli O157:H7, Shigella); Francisella tularensis (tularemia); Glanders (Burkholderia mallei); Melioidosis (Burkholderia pseudomallei); Plague (Yersinia pestis); Psittacosis (Chlamydia psittaci); Q fever (Coxiella burnetii); Ricin toxin from Ricinus communis (castor beans); Rickettsia prowazekii (typhus fever); Salmonella species (salmonellosis); Salmonella Typhi (typhoid fever); Salmonellosis (Salmonella species); Shigella (shigellosis); Shigellosis (Shigella); Smallpox (variola major); Staphylococcal enterotoxin B; Tularemia (Francisella tularensis); Typhus fever (Rickettsia prowazekii); Variola major (smallpox); Vibrio cholerae (cholera); Viral encephalitis (alphaviruses [e.g., Venezuelan equine encephalitis, eastern equine encephalitis, western equine encephalitis]); Viral hemorrhagic fevers (filoviruses [e.g., Ebola, Marburg] and arenaviruses [e.g., Lassa, Machupo]); Water safety threats (e.g., Vibrio cholerae, Cryptosporidium parvum); bacterial responsible for necrotizing fasciitis; methicillin-resistant Staphylococcus aureus (MRSA); vancomycin-resistant Enterococcus (VRE); bacteria or mycoplasma causing pneumonia; bacteria responsible for nosocomial infections; and Yersinia pestis (plague).

Common household molds that can be remediated using systems of preferred embodiments include, but are not limited to Acremonium; Alternaria; Aspergillus fumigatus; Aspergillus niger; Aspergillus species Var. 1; Aspergillus species Var. 2; Aureobasidium; Bipolaris; Chaetomium; Cladosporium; Curvularia; Epicoccum; Fusarium; Geotrichum; Memnoniella; Mucor; Mycelia sterilia; Nigrospora; Paecilomyces; Penicillium species Var. 1; Penicillium species Var. 2; Pithomyces; Rhizopus; Sporothrix; Sporotrichum; Stachybotrys; Syncephalastrum; Trichoderma; and Yeast. Molds need high humidity levels and a surface on which to grow. Common areas for mold growth are garbage containers, food storage areas, upholstery, and wallpaper. Molds also commonly grow in damp areas such as basements, shower curtains, window moldings, and window air conditioners.

Indoor allergens that can be remediated by using systems of preferred embodiments include dust mite feces. Dust mite feces are the major source of allergic reaction to household dust. The mites thrive on shed human skin and are most commonly found in bedrooms, where skin cells are abundant. Preventive measures include frequently laundering bed linens in hot water and removing carpets from the room. In some cases, homeowners might have to encase the bed mattress, box springs, and pillows in vinyl covers. Other allergens of animal origin include skin scales shed from humans and animals. Commonly called dander, these are another major allergen. Dander from such animals as cats, dogs, horses, and cows can infest a home even if the animal has never been inside. Rodent urine from mice, rats, and guinea pigs is another allergen. Cockroach-derived allergens come from the insect's discarded skins. As the skins disintegrate over time, they become airborne and are inhaled.

The systems of preferred embodiments can also have utility in treating for bed bugs, fleas, ticks, mosquitoes, flies, spiders, ants, cockroaches, and other insects.

Tobacco smoke, engine exhaust, and similar allergens and odors or odor-causing agents can be abated using systems of preferred embodiments, as can volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like. VOCs include organic solvents, certain paint additives, aerosol spray can propellants, fuels (such as gasoline, and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone.

Odors and odor-causing substances that can be abated include skunk odors, urine, pet odors, and the like.

It is generally preferred to subject the substance to be abated or remediated to ozone at the preferred concentrations in the atmosphere as discussed below, generally 2 to 10 ppm, and adjust the length of treatment as necessary to ensure satisfactory kill and/or neutralization levels. Serious mold infestations are generally the most resistant substance to remediate. Treatment times of 1, 2, 3, 4, 5, 6, 12, 24, or 48 hours or more can be employed to ensure penetration of the ozone/hydroxyl mixture throughout the entire mass of a serious infestation and achieve a 100% kill and neutralization. However should it be difficult to have the contaminated premises vacated for these long periods of time, it may be necessary to leave the treatment time at the preferred treatment time (2 to 4 hours) while at the same time increasing the level of ozone and/or hydrogen peroxide. Ozone levels of 20, 30, 40, 50, 100, 200, 400 or more ppm can be desirable along with a proportionate increase of hydrogen peroxide to such that the concentration (by weight) of hydrogen peroxide in the atmosphere is up to about 150% or more of that of ozone in the atmosphere introduced into the area to be treated, preferably from about 75% to about 150%, more preferably from about 85% to about 125%, and even more preferably from about 90% to about 100%. However, in certain embodiments it can be desirable to provide even higher concentrations of ozone.

A treatment time of 2 to 3 hours when using systems of preferred embodiments is generally effective in abating serious mold infestations. However, an individual mold spore is generally killed and neutralized within minutes. Protein based allergens are generally neutralized within minutes. Bacteria are generally killed after an exposure time of minutes or less. Viruses are generally killed after an exposure of less than a minute, typically after exposure times as short as several seconds. Certain molds, bacteria, allergens, and viruses can be more resistant to ozone treatments than others. For example, anthrax spores have a hard coating that is preferably “softened up” by exposure to humidity prior to ozone treatment to ensure that all spores are destroyed by a subsequent ozone treatment. See, e.g., R. G. Rice, Ozone Science and Engineering, Vol. 24. pp. 151-158 (2002). In using systems of preferred embodiments, it is generally preferred to employ treatment times of 2 to 3 hours, since such times are generally satisfactory for abating a mold infestation, and well exceed the lower limit of treatment time for substances such as protein-based allergens, bacteria, and viruses. However, when it is desired to abate a particularly virulent pathogen, such as anthrax, it can be desired to employ a treatment time of over 3 hours, for example, a treatment having a duration of 5, 6, 7, 8, 9, 10, 11, 12, 16, 20, 24, 28, 32, 36, 40, 44, 48 hours or more. It is also desirable, when abating a particularly virulent pathogen, to also increase the level of ozone and/or hydrogen peroxide. Ozone levels of 20, 30, 40, 50, 100, 200, 300, or 400 ppm or more can be desirable and the concentration by weight of hydrogen peroxide can be increased to more than 100%, preferably more than 125%, and most preferably more than 150% of the concentration by weight of ozone introduced in the area to be treated. Any suitable combination of increased time, increased relative concentration of hydrogen peroxide, and/or increased ozone concentration can be employed.

The use of systems of preferred embodiments is generally successful in abating substances that are sourced indoors, for example, a mold infestation, dander from a companion animal living in a house, mite feces, tobacco smoke, volatile organic compounds from newly installed carpeting or freshly painted walls, and the like. Substances from outside sources, such as pollen, automobile or diesel exhaust, and the like, can also be treated, but recurring treatments can be necessary as such substances reenter the interior space from outside.

### Areas and Materials That Can Be Treated

Any interior or contained space is amenable to treatment using systems of the preferred embodiments. For example, single family homes, apartment buildings, office buildings, schools, hospitals, post offices, locker rooms, restaurants, ships, trains, buses, airplanes, trucks, recreational vehicles, mobile homes, manufactured houses, cargo containers, and the like are particularly well-suited to treatment. The systems are particularly well suited for use in newly constructed homes, buildings, vehicles, and the like, which generally contain substantial quantities of VOC sources, such as newly-installed carpeting and flooring, fresh paint, adhesives, and the like. Larger enclosed spaces, such as warehouses, barns, chicken houses, and other buildings housing farm animals, grain elevators, factories, hangars, subway systems, air terminals, and the like, can also be treated provided that the preferred levels of ozone, hydrogen peroxide, temperature, and humidity can be attained. In certain embodiments, rather than seal and treat the entire volume of enclosed space, the space can be partitioned so as to maintain the preferred levels of ozone, hydrogen peroxide, temperature, and humidity in areas adjacent to those to be treated. For example, plastic sheeting can be draped over a floor or wall to be treated so as to contain the ozone/hydrogen peroxide and humidity and maintain the temperature adjacent to the treated area.

The systems of preferred embodiments can also be employed to treat materials. Materials that can be treated include any materials that can tolerate exposure to the ozone, hydrogen peroxide, humidity, and temperature conditions of preferred embodiments without suffering damage. For example, clothing, bedding and linens, rugs, mail, packages, documents, furniture, food items, agricultural products such as seeds, grains, cut flowers, produce, fruits vegetables, and live plants, containers and packaging materials, and the like. A suitable chamber can be constructed that can be sealed to maintain conditions of ozone and hydrogen peroxide concentration, humidity and temperature at preferred levels, and the material placed inside that chamber and subjected to treatment. In an automated process, materials can be moved through an airlock and into the chamber for treatment for a suitable time period, and then moved out of the chamber through another airlock. Such automated processes can be particularly well suited for the decontamination of large volumes of mail for pathogens such as anthrax, or the decontamination of animal carcasses or meat products (beef, pork, poultry, seafood, and the like) for pathogens such as salmonella or e. coli. If the treatment chamber is of sufficient size, vehicles such as passenger cars or trucks hauling various cargo, rail cars, and the like can be treated therein.

In another embodiment, it can be preferred to subject a room or space to periodic decontamination, such as a surgical suite in a hospital, a treatment or waiting room in a clinic, a kitchen or a restaurant, a bar or nightclub, a theater, a bingo hall, a meat processing area of a grocery store, or the like. In such embodiments, it is generally preferred to permanently install equipment in a location adjacent to the space to be treated. Such equipment can include a control unit, security devices, an oxygen concentrator, an ozone generator, an ultrasonic humidifier, a hydrogen peroxide source, a heater and/or air conditioner, and a ventilation unit. Prior to treatment at a convenient time (for example, after work hours), the space is scanned to ensure that no personnel are present in the room. Motion detectors, heat detectors, video cameras and the like can be suitable for such purposes. Once the space has been confirmed to contain no personnel, a lock down procedure is instituted to prevent anyone from entering the space during the treatment and to maintain conditions within the space. Treatment is then conducted according to preferred embodiments. After ozone levels have been reduced to acceptable levels, the space is then unlocked. If it is necessary to reduce ozone levels to acceptable levels in rapid fashion, ozone destruct units can be employed. A computer can be employed to control the lockdown and treatment process, as well as the treatment schedule.

### Assessment of Conditions

In preferred embodiments, an assessment of conditions in the premises is typically conducted to determine, e.g., pathogen, allergen, or gas levels, followed by treatment with ozone/hydrogen peroxide. An assessment is typically conducted to discover if a premises (e.g., house, office building, boat, and the like) has a pathogen, allergen, or other problem that can be eliminated by using the abatement systems of preferred embodiments. If it is determined that the problem can be effectively eliminated, abatement can be conducted. It is also recommended that the underlying problem responsible for the mold infestation be identified and eliminated, so as to prevent future infestations. As part of the assessment, mold tests, e.g., tests for specific types of mold can be conducted. Other testing can include tests for VOCs, tests for allergens, tests for pathogens, and the like. Ambient conditions, including temperature and relative humidity, the size of the area to be treated (square footage, volume), can also be measured. During the assessment process, it is preferred to wear appropriate protective gear, e.g., respirators, ear plugs, gloves, foot coverings, clothing coverings, goggles, and the like. For example, when dealing with an extensive infestation of particularly toxic mold, it is generally preferred to wear full hazardous material protective gear. In situations wherein the premises are subject to odors that are unpleasant but not otherwise harmful, a respirator, or no protection at all can be sufficient.

While assessments are typically conducted, in certain embodiments an assessment may not be necessary. For example, when an obvious mold infestation is present, when elimination of odors or allergens is the major impetus behind the treatment, or when the premises are treated on a periodic basis for chronic conditions such as asthma triggered by dust mites, the treatment can be initiated without performing any prior assessment.

### Preparations Before Treatment

Before commencing an abatement process or other process according to preferred embodiments, it is preferred to determine the area or volume of the premises to be treated such that the target dosage, the quantity, and type of treatment materials and equipment that is sufficient to complete the abatement process can be determined.

It is preferred to meet with the owner (or occupier) of the building before commencing the abatement process. During the meeting, the process can be explained and the owner can assist in preparations for the abatement process. For example, all individuals, unless provided with appropriate protective gear, are instructed to leave the premises for the duration of the abatement process. Any animals, such as pets, are removed from the premises, and it is preferred to remove plants. It is not necessary to remove fish. Problem areas can be identified for treatment, along with areas that may not be amenable to treatment using the systems of preferred embodiments, or areas wherein a contamination can reoccur if the source of the contamination is not eliminated. Areas not amenable, due to either the location and/or extent of the infestation, can necessitate more extensive treatment or remediation steps, such as those employed to remove and/or dispose of toxic waste, e.g., procedures similar to those used in asbestos abatement.

### Pretreatment

In certain embodiments, it can be desirable to pre-treat the area prior to subjecting the area to ozone treatment. A preferred pretreatment involves subjecting the area to be treated with humidity. Certain pathogens, such as mold spores and anthrax, are resistant to conventional ozone treatment. The anthrax bacterium, for example, possesses a hard shell that resists penetration by ozone. By subjecting anthrax to humidity prior to ozone treatment, the ozone, and/or hydroxyl is better able to penetrate the microorganism and destroy it. Likewise, mold spores are resistant to penetration by ozone, but can be made more amenable to treatment by first subjecting them to humidity. Treatment of VOCs is also facilitated by humidity. Ozone reacts with the atmospheric water and hydrogen peroxide to produce reactive hydroxyl groups, which then react with certain VOCs to yield less harmful or harmless substances.

When the material to be treated includes molds, a pretreatment consisting of exposure to a relative humidity of 70% to 95% is typically employed. Typical pretreatment exposure times of 10 minutes, 15 minutes, 30 minutes 45 minutes, 1 hour, 1.5 hours, 2 hours, 2.5 hours, 3 hours, 4 hours, or more can be employed, depending upon the substances to be abated and the nature of the space or material to be abated. In residential mold remediation, pretreatment times of from 30 minutes to 2 hours are generally preferred. In decontaminating a material infested with anthrax, pretreatment times of from 12 hours to 24 hours are generally preferred.

### Abatement of Living and Working Spaces

If the area to be treated has an air duct system (e.g., heating or heating/air conditioning system), it is preferred to position one or more ozone generators and humidifiers adjacent to the return air inlet. Typically, for treating volumes of 25,000 cu. ft. or less, it is preferred that at least 10 g/hr of ozone is drawn into each air inlet. However, in certain embodiments satisfactory results can be obtained at a lower level of ozone generation, for example. at about 0.1, 0.5, 1, 2, 3, 4, 5, 6, 7, 8, or 9 or more g/hr. Likewise, in certain embodiments a higher level of ozone generation can be preferred, for example, about 10, 11, 12, 13, 14, 15, 20, 25, 30, 40, 50, 60, 70, 80, 90, or 100 or more g/hr. Along with the higher level of ozone generation is a proportionately higher level of hydrogen peroxide introduction, generally at a concentration by weight that is 75% to 150% of concentration by weight of ozone provided. Commercially available ozone generators are available in a variety of sizes. Size is generally reported in terms of ozone output in grams/hour. It is generally preferred to locate mid-sized (e.g., 6. 10 or 15 gm/hr) ozone generators in larger areas, e.g., living room, kitchen/family room, open stairways, open office spaces, and the like. Smaller ozone generators (e.g., 1-5 gm/hr) are preferably situated in small or closed-in areas, or areas that are unlikely to get circulating air, for example, basements, storage areas, and the like. Generally dosage calculations show that ozone generators that are capable of generating 60 g/hr of ozone are sufficient to treat a 2,000 sq. ft. house with forced air ducting. More ozone can be preferred for a house without forced air ducting. Typically, dosage calculations show that one gram of ozone per hour is preferably delivered for every 250 cu. ft. of area to be treated. More or less ozone can be desirable, depending on humidity, temperature, and specific condition being treated. Therefore, for each 1 gm/hr of delivered ozone, an area of 33 sq. ft. with 8 ft ceilings can be decontaminated. Determination of the amount of ozone required is made according to the target dosage found in the dosage tables relating time of treatment to the ozone concentration, delivered hydrogen peroxide, humidity and temperature to the problem be treated such as mold, allergens, pathogens or VOCs.

After the ozone generators are situated and turned on, the forced air system is then turned on to circulate the air. It is generally preferred that no heating or cooling of the air is conducted, however, in certain embodiments it can be preferred to heat or cool the air so as to obtain optimal abatement results.

Humidity is a significant factor in the kill rate of allergens and pathogens. In general, the higher the humidity, the faster the ozone kills the pathogen or destroys the allergen. While not wishing to be bound to any particular theory or mechanism, it is believed that the ozone reacts with the water vapor forming hydroxyl radicals thereby increasing the effectiveness of the process. Generally, it is preferred that the relative humidity in the premises to be treated be at least 30% or more, preferably the relative humidity is at least 40%, 45%, 50%, 55%, or 60% or more, more preferably the relatively humidity is from 65% to about 70%, 75%, 80%, 85%, or 90% and most preferably the relative humidity is from about 70% to about 90% or 95%. Relative humidities greater than 95%, especially relative humidities of 100%, are generally not preferred due to the risk of condensation, which can lead to bleaching of sensitive materials. However, in certain embodiments relative humidities greater than 95% can be acceptable if sensitive materials are not a concern. In order to ensure that there is adequate formation of hydroxyl radicals, specifically at low humidity levels, hydrogen peroxide is preferably supplied to the area being treated at a concentration by weight that is up to about 150% or more (preferably about 75% to about 150%) of the concentration by weight of ozone being delivered. For example, if the area is being treated with about 60 g/hr of ozone, then from about 45 to 90 g/hr of hydrogen peroxide is also delivered. At low humidities, and for the abatement of particularly virulent pathogens, it is generally preferred to employ higher relative concentrations of hydrogen peroxide, preferably greater than 100%, 125%, or 150% or higher.

In coastal areas, ambient humidity can provide optimal results. In these situations the ultrasonic humidifiers are used only to deliver a minimal amount of hydrogen peroxide. However, in desert areas or under low humidity conditions (e.g., winter in northern areas of the United States), it can be preferred to increase the humidity via one or more ultrasonic humidifiers so as to achieve optimal results. Once treatment is completed, it may be desired to employ one or more dehumidifiers to rapidly restore the ambient humidity to the treated premises, if the premises are humidity-controlled. In certain instances, it may not be feasible to humidify the area to be treated. For example, the area can contain humidity-sensitive materials (e.g., antiques, rare books, old documents, fragile textiles or wallpaper and the like). In those instances, treatment can be conducted under ambient humidity conditions with the addition of hydrogen peroxide, but the duration of the ozone treatment can be extended to ensure satisfactory results. However under these conditions it can be desirable to increase the ratio of delivered hydrogen peroxide to ozone from 150% to 200%, 250%, 300%, 350%, 400%, or even 1000% or more of the concentration by weight of ozone present. Any suitable schedule of introducing ozone, moisture, and/or hydrogen peroxide can be employed, e.g., constant introduction of one or more of ozone, moisture, and hydrogen peroxide, intermittent introduction of one or more of ozone, hydrogen peroxide, and humidity, varying concentrations, varying temperatures, and the like.

Temperature levels also correlate with the effectiveness of the treatment method in killing mold. Generally, as temperature increases, the effectiveness of the treatment increases. However the amount of ozone required to achieve and maintain the target dosage also increases as the ozone more readily reverts back to oxygen at higher temperatures (i.e., ozone exhibits a shorter half life at higher temperatures). Generally, it is preferred to conduct the treatment at a temperature typically considered a “room temperature,” namely about 17.7° C. (64° F.), 18.3° C. (65° F.), 18.8° C. (66° F.), 19.4° C. (67° F.), 20° C. (68° F.), 20.5° C. (69° F.), or 21.1° C. (70° F.) up to about 21.6° C. (71° F.), 22.2° C. (72° F.) 22.7° C. (73° F.), 23.3° C. (74° F.), 23.8° C. (75° F.), 24.4° C. (76° F.), 25° C. (77° F.), 25.5° C. (78° F.), 26.1° C. (79° F.), 26.6° C. (80° F.), 27.2° C. (81° F.), 27.7° C. (82° F.), 28.3° C. (83° F.), 28.8° C. (84° F.), or 29.4° C. (85° F.). In most residential and commercial settings, the ambient temperature falls within this range. However, if the premises to be treated is not equipped with heating or air conditioning, it can be preferred to adjust the interior temperature prior to initiating treatment, or to control the temperature at a pre-selected level during treatment. When the ambient temperature is high and the structure to be treated is not equipped with air conditioning, an air conditioning unit can be provided as part of the equipment system and used to cool the temperature, e.g., down to below 29.4° C. (85° F.). Cooling the interior to below 17.7° C. (64° F.) generally results in only an incremental reduction in the rate of ozone decomposition. Thus, it is generally not preferred to cool the interior below this temperature. If the ambient temperature is substantially below 17.7° C. (64° F.), it is generally preferred to heat the interior. In certain conditions, the temperature in the structure to be treated can be controlled to a pre-selected temperature, for example, a cold storage locker or a room containing equipment or machinery that must be operated at an elevated or reduced temperature. Under such conditions, the treatment is preferably conducted at ambient temperature and the ozone level and/or humidity is adjusted to achieve optimum results. In certain embodiments, however, it can be desirable to treat an area at temperatures outside of those typically considered ambient temperatures. For example, a refrigerated unit maintained at a temperature above 0° C. (32° F.) can be satisfactorily treated by adjusting the humidity, hydrogen peroxide, and ozone levels. Generally, ozone levels are increased at low temperatures. However, lower ozone levels of 2 to 10 ppm can be employed in conjunction with a longer treatment time.

Ozone levels of 2 to 10 ppm are generally preferred for treating mold and other contaminants. However, in certain embodiments it can be preferred to employ ozone levels of 1.9, 1.8, 1.7, 1.6, 1.5, 1.4, 1.3, 1.2, 1.1, 1.0, 0.9, 0.8, 0.7, 0.6, 0.5, 0.4, 0.3, 0.2 ppm or less. In other embodiments, ozone levels of 11, 12, 13, 14, 15, 16, 17, 18, 19, 20, 25, 30, 35, 40, 45, 50 ppm or more can be preferred. At optimal hydrogen peroxide, humidity, and temperature levels, a longer treatment time is preferably employed at reduced ozone levels and a shorter treatment time is preferably employed at higher levels so as to ensure a satisfactory kill and/or neutralization level.

In terms of the optimal combination of ozone level, hydrogen peroxide, temperature, and humidity, it is generally preferred to conduct a treatment at a temperature of about 21.1° C. (70° F.), a relative humidity of about 85%, a hydrogen peroxide addition of 107% by weight of ozone, and an ozone level of about 10 ppm. Under these conditions, the length of time required to achieve a 100% kill for mold spores is minimized. For in a typical residential setting, a 100% kill can be achieved in about 3 hours or less. When the temperature ranges from about 15.5° C. (60° F.) to about 26.6° C. (80° F.), i.e., interior temperatures typically observed for residential and commercial buildings, it is preferred that the relative humidity be in the range of about 70% to about 85%, and the ozone levels be in the range of about 2 ppm to about 10 ppm (with a corresponding hydrogen peroxide concentration by weight of 75% to 150% of that of the ozone concentration by weight). Under these conditions, the optimal time to achieve a 100% kill is typically about 1 to about 3 hours.

For structures situated in high humidity environments, e.g., the coastline, the Midwest during summer, and the like, wherein the relative humidity ranges from about 85% to about 95% and ambient temperatures range from about 21.1° C. (70° F.) to about 32.2° C. (90° F.), lower ozone levels can be employed. Under these conditions, the optimal time to achieve a 100% kill is typically about 1 to about 3 hours.

For structures situated in low humidity environments, e.g., desert communities, and the like, wherein the relative humidity ranges from about 5% to about 20% and ambient temperatures range from about 23.8° C. (75° F.) to about 37.7° C. (100° F.), it is preferred that the relative humidity is raised via the use of an ultrasonic humidifier or other suitable method to from about 70% to about 85% before beginning treatment and that the ozone levels are in the range of about 2 ppm to about 10 ppm. Under these conditions, the optimal time to achieve a 100% kill and/or neutralization is typically about 1 to about 3 hours. If it is not feasible to raise the humidity levels to the 70% to 85% range, then a higher level of hydrogen peroxide can be introduced to ensure the production of sufficient hydroxyl radicals to complete the decontamination process. Hydrogen peroxide levels of 125% to 150% of the weight of introduced ozone can generally be used to ensure decontamination. However under the most extreme conditions it may be desirable to increase the ratio of delivered hydrogen peroxide to ozone from 150% to 200%, 250%, 300%, 350%, 400%, or even 1000% or more.

In commercial and residential settings, and the like it is preferred to open all closet and cupboard doors, and to move items stored in cupboards and closets to facilitate air circulation. Doors, windows, fireplace dampers, and other air egresses are preferably closed.

In residential settings, if dust mites are problematic, it is preferred that all linens be taken off beds and washed in 60° C. (140° F.) water. Mattresses are typically removed from the box spring and leaned up against the box spring so as to facilitate air circulation around the mattress. If feasible, blankets, pillows, and bed spreads are preferably placed in a manner that allows satisfactory air circulation. Exhaust fans, e.g., in the kitchen, bathroom, and the like, are turned on, which helps ensure that ozonated air reaches the outlet ducting of these areas. If such fans have a variable speed, they are preferably operated at their lowest possible level to reduce the amount of ozone that will evacuated while at the same time ensuring that the vent system is decontaminated.

Before the ozone generators are activated, ultrasonic humidifiers may be employed to achieve required relative humidity levels and to deliver the hydrogen peroxide in prescribed amounts according to the target dosage. Once target humidity levels are achieved, the area can be evacuated of any nonessential personnel. For those individuals remaining in the premises, a respirator (and goggles if the respirator is not a full-face respirator) is preferably in place before turning on the ozone generators. The ozone generators and humidifiers supplying hydrogen peroxide are typically turned on starting with the most remote areas of the premises and finishing with the heating and air conditioning inlet or inlets last. After the ozone generators have operated for a short time period, typically about ten minutes, it is preferred to test and record ozone, temperature, and humidity levels. For non-automated ozone generators this will require reentering the premises and taking the necessary readings. Once the ozone and humidity levels have reached target dosage levels, typically at least about 2 to 10 ppm ozone and 50-90% RH, effective treatment has begun, and the premises can be left closed for the duration of the treatment. Although 2 to 10 ppm ozone is generally preferred, in other embodiments a higher or lower ozone level can be desirable, e.g., less than 0.1, 0.5, 1, 2 ppm ozone up to about 15, 20, 30, 40, 50, 60, 70, 80, 90, 100, 500, 1000, 2000, 3000, 4000, 5000 ppm ozone or more. For these higher levels of ozone, proportionately higher levels of hydrogen peroxide are also generally preferred, again based on the 75% to 150% hydrogen peroxide to ozone by weight. If the contamination is particularly extensive or the mold to be abated particularly toxic, higher ozone levels can be preferred. To prevent injury, all doors and other possible entrances to the building are preferably locked and caution signs placed the entrances.

Humidity, temperature, and ozone concentration readings are typically recorded in each room during the course of the treatment procedure. These recordings can be obtained automatically, e.g., by stand-alone recorders in the rooms or by the ozone generator equipped with measuring devices. Alternatively, the recordings can be obtained manually at pre-selected intervals.

Treatment is typically continued for up to 48 hours depending on the target dosage which relates temperature, humidity, ozone concentration, and condition being treated to treatment time. After treatment is completed, the ozone generators and humidifiers are turned off and ozonated air can be evacuated from the premises if immediate occupancy is desired. Alternatively the ozone can be left to degrade back to oxygen if immediate occupancy is not required. If immediate occupancy is desired fans are typically placed in one or more doorways and/or windows to blow ozonated air out of the premises. High volume fans (9400 cfm.) are generally preferred for residential applications; however fans capable of moving more air or less air than 9400 cfm. can also be suitable for use. The evacuation fans are typically operated for 30 minutes, and then ozone levels are tested, especially in areas where circulation from the fans is lowest, such as bedrooms, basement, and the like. Depending upon the circulation efficiency, longer or shorter operation times can be preferred.

As an alternative to the use of evacuation fans, the premises can remain closed and the ozone can dissipate and/or decompose to safe levels without taking active steps to remove ozonated air from the premises. In an enclosed space with poor air circulation, ozone levels will typically return to ambient levels after about 6 hours. However, it is generally preferred to take active measures to evacuate ozonated air such that the delay in reoccupying the premises is minimized.

Ozone levels are periodically tested until a pre-selected ozone level at which it is safe to reoccupy the building is achieved. A self contained breathing apparatus or respirator is preferably employed for testing until a level of 0.1 ppm ozone, 0.05 ppm ozone, or less is recorded, or until levels of ozone similar to outside ambient levels are achieved, if ambient levels are higher than 0.1 or 0.5 ppm. An ozone level of 0.05 ppm has been determined by the FDA to be a safe level for continuous exposure. An ozone level of 0.1 has been determined by OSHA to be safe for exposure times of up to 8 hours. Once the level drops below 0.05 ppm or outside ambient levels, the treated area is typically safe for reoccupation by people, animals, and plants and treatment is complete. If level is not below 0.05 ppm in all areas, circulation by fans is continued until this level is reached. While 0.05 ppm is the preferred level deemed safe for reoccupation, in certain embodiments it can be preferred that a lower level be attained, e.g., a level characteristic of ambient ozone levels prior to treatment. Alternatively, a higher level of ozone can be acceptable in certain embodiments.

Ozone levels can be tested using commercially available instrumentation, such as that manufactured by Eco Sensors of Santa Fe, N.M. When testing for current ozone levels, the instrument is used in accordance with the manufacturer's instructions. These typically include allowing adequate warm up time (generally at least 5 minutes), not blocking the air flow into the instrument while testing, making all measurements in still air as moving air can affect the readings, keeping the instrument away from the body as body odors can bias the reading, not using the instrument to take measurements directly from the outlet of the ozone generators which can result in incorrect readings and/or damage to the instrument.

After the premises has been deemed safe for reoccupation, doors and windows can be unlocked, caution signs can be removed, and all equipment, including fans, ozone generators, and humidifiers can be removed from the premises.

It is noted that in the case of allergens and pathogens, treatment does not remove the allergen or pathogen from the treated area. In the case of a pathogen, such as mold spores, the organism is killed. If the pathogen has an ability to produce an allergic reaction, this ability is also neutralized. In the case of an allergen such as animal dander or dust mite feces, the protein causing the allergic reaction is neutralized by the ozone treatment such that it is unable to cause an allergic reaction. A subsequent cleaning step to remove the dead organism or deactivated allergen can be desirable in certain embodiments, but is not necessary.

### Pretreatment and Post-treatment for Ozone Control

After an ozone treatment is administered and ozone returns to ambient levels, a strong ozone odor can still be noticeable. If there is such an odor, it is generally associated with closets and bedrooms. While not wishing to be bound to any particular theory, it is believed that fabrics or other materials containing natural or synthetic fibers having an electrostatic charge can attract and hold ozone, slowly releasing it back into the surrounding air at safe but noticeable levels. While a large portion of the population considers ozone to have a pleasant odor, some individuals consider the odor unpleasant. Other individuals, typically those suffering from asthma, can find ozone to be an irritant. With time, any ozone present will eventually dissipate without the need for specific measures; however, a method for reducing or eliminating lingering ozone or the odor associated with ozone is desirably employed in certain embodiments.

Any suitable method can be employed for destroying or removing lingering ozone. For example, ultraviolet (UV) light is preferably employed. A UV light source can be brought into the space treated, and the light therefrom can be passed over the materials that are holding the ozone, such as bedding, clothes, drapes, and the like. The UV treatment is preferably conducted after completion of the ozone treatment, most preferably as soon as detected ozone levels reach ambient levels.

Removal of ozone can also be accelerated by subjecting the interior spaces to elevated temperatures, for example, by a radiant heater or hot air blower.

In other embodiments, it can be preferred to employ ions. Depending upon the nature of the materials in the treated space, it can be desirable to employ only positive or negative ions, or to employ positive ions for a time followed by negative ions, or vice versa. Any suitable equipment for generating ions can be employed. It is generally preferred to employ an ion generator capable of producing 1×10¹² ions per second (negative or positive). Such an ion generator is generally suitable for use in rooms or spaces having an area of approximately 500 sq. ft. Alternatively, bipolar ionization can be employed. Bipolar ionization uses an alternating current to produce both positive and negative ions. Bipolar ionization utilizes a process involving association and disassociation to generate a highly reactive mixture of ionized gas consisting of atoms, molecules, and free radicals capable of creating chemical changes. There are several types of devices that can be used for this process. For HVAC applications, a non-thermal type of surface discharge reactor is preferably used. Bipolar ionization was first used commercially in 1972 in the food and meat industry in Western Europe to improve shelf life of perishable foods with limited or no mechanical refrigeration.

When the bipolar ion generator is connected to an oscilloscope, a sinusoidal waveform is observed. On one side of the waveform, the bipolar generator produces positively charged ionized gas molecules and on the other side of the waveform, the bipolar generator produces negatively charged ionized gas molecules. This is a pulsed AC system, which alternately produces negative and positively ionized gas molecules. In operation, a pulsed ion field is created in the vicinity of the bipolar generator. As air passes through the ion field, electrons in the valence shells of stable molecules receive excitation energy. As the air stream moves out of the ion field and through the air-handling unit, the electron vibrational energy permits valence electrons to overcome nuclear attraction and escape. Chemical bonds are broken in gas molecules, ionic compounds disassociate to positive and negative ions, and covalent compounds disassociate to free radicals. In the absence of a polar field, the highly unstable ions and free radicals combine to form more stable compounds.

To determine what type or types of ions are preferred for treating a space, the materials contained within the space can be classified on the basis of their place in a triboelectric series. Below is a very short triboelectric series that provides an indication of the ordering of some common materials. A material that charges positive will be the one that is closer to the positive end of the series and the material closer to the negative end will charge negatively. Accordingly, to reduce the charge on the material, ions of opposite polarity can be applied. It is the work function of the material that determines its position in the series. In general, materials with higher work function tend to appropriate electrons from materials with lower work functions. Triboelectric series (from positive to negative): positive (+)>asbestos>glass>nylon>wool>lead>silk>aluminum>paper>cotton>steel>hard rubber>nickel & copper>brass & silver>synthetic rubber>Orlon>saran>polyethylene>Teflon>silicone rubber>negative (−). While such triboelectric series can be helpful in determining the preferred ion treatment, other factors can affect the preferred treatment. For example, real materials are seldom very pure and often have surface finishes and/or contamination that strongly influence triboelectrification. The spacing between materials on a triboseries does not allow one to predict with any confidence the magnitude of the charge separated. Many factors besides the difference in the electronic surface energy, including surface finish, electrical conductivity, and mechanical properties, can also strongly influence results.

In addition to controlling ozone odor, bipolar ionization methods can yield additional benefits, including microbiological control, control of other odors and gas phase chemicals, static control, and filtration enhancement. After dissipating the ionization energy, air with a balanced electrical charge remains. In the absence of any electrical charge, submicroscopic particulates are not attracted to foreign surfaces and remain airborne and naturally buoyant. Air currents established by an efficient air distribution system displace the particulates and carry them back to the filters in the air-handling unit. Particulates that pass through the filters remain buoyant on subsequent circulation cycles and are returned to the filters for another attempt at removal. With every pass through the filters, the probability increases for removal.

It is generally preferred to employ an ion treatment during the ozone treatment. However, an ion treatment can also be conducted before or after the ozone treatment, or at any suitable time.

### Remediation of Norwalk Virus

Interior spaces, such as in cruise ships, infected with Norwalk virus can be remediated using systems of preferred embodiments. The procedure generally preferred is as follows: Ships are generally constructed such that sections of the ship can be isolated from the remainder of the ship. These sections have independent heating, ventilating. and air conditioning systems (HVAC) as well as sealable doors providing total isolation. Humidification and ozonation equipment can be brought aboard the ship and placed within one or more of the isolatable sections. The ultrasonic humidifiers and ozone generators can be placed near or within the air inlets of the HVAC system and all systems placed in operation. This circulates the ozone, hydrogen peroxide, and humidity throughout the isolated section of the ship at target dosages determined to be lethal to the Norwalk and other viruses and bacteria. Once the target dosage has been achieved then the ozone can be left to decompose back to oxygen or it can be evacuated by opening the outside makeup air system to allow 100% makeup air, thereby evacuating the ozone and allowing rapid reoccupation of the treated section.

### Remediation of Anthrax

Systems of preferred embodiments are suitable for use in decontaminating indoor areas or materials contaminated with anthrax. The procedure generally preferred is similar to that noted for Norwalk virus, with the exception that the area to be treated is pre-treated with humidity for a period of 12 to 24 hours at levels of humidity greater than 70%. Ultrasonic humidifiers are placed within the area to be treated. They are turned on and allowed to operate until such time as a level of at least 90% relative humidity is achieved. Once this level has been achieved, the pretreatment period has begun. Once the pretreatment period has been concluded, the actual treatment can be completed based on target dosages for anthrax. Target dosages for anthrax require that the amount of hydrogen peroxide relative to ozone introduced via the ultrasonic humidifiers to the treatment area is higher than in other decontamination situations, preferably at least from about 125 to 150%, but in certain embodiments even higher. With regard to safety issues, significantly more stringent safety procedures are required when treating an area contaminated with anthrax or other particularly virulent pathogens. Scientifically accepted hazardous material safety procedures are followed strictly by all personnel involved in the decontamination procedure.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1.-87. (canceled)

88. An ozone generator device, comprising:
a housing, comprising:
an air inlet, and
an air outlet;
a lamp within the housing, wherein the lamp is configured to emit ozone generating light, and wherein the lamp comprises:
a glass enclosing a gas,
one or more pins, configured to electrically connect to a power source,
a filament within the glass, and
first and second electrodes extending through the glass and electrically connected to the one or more pins, wherein the filament is electrically and mechanically connected to the first and second electrodes; and
a fan configured to force air from the air inlet out of the device through the air outlet, wherein the air exiting the device includes ozone.

89. The device of claim 88, wherein the filament is medium gauge.

90. The device of claim 88, wherein the filament comprises first and second ends and the filament is connected to the first and second electrodes at the first and second ends, respectively.

91. An air treatment system, comprising:
an ozone generator comprising communication circuitry; and
a humidifier comprising communication circuitry,
wherein the ozone generator and the humidifier are configured to communicate with each other.

92. The system of claim 91, wherein the humidifier is configured to diffuse hydrogen peroxide into the air.

93. The system of claim 91, wherein the ozone generator and the humidifier are configured to communicate status information.

94. The system of claim 91, wherein at least one of the ozone generator and the humidifier comprises a sensor, and is configured to communicate sensor information.

95. The system of claim 91, wherein at least one of the ozone generator and the humidifier is electrically connected to communication circuitry external thereto.

96. The system of claim 91 further comprising a communication hub, configured to communicate with at least one of the ozone generator and the humidifier.

97. The system of claim 96, wherein the communication hub is further configured to communicate with another communication hub of a separate system.

98. The system of claim 96, wherein the communication hub is further configured to communicate with equipment located in a building remote to the system or in a service vehicle.

99. A method of treating air, comprising:
providing an ozone generator with communication circuitry;
providing a humidifier with communication circuitry;
diffusing hydrogen peroxide into the air with the humidifier;
sending a first signal from the ozone generator to the humidifier; and
sending a second signal from the humidifier to the ozone generator.

100. The method of claim 99, wherein the first signal comprises status information.

101. The method of claim 99, wherein the second signal comprises status information.

102. The method of claim 99, wherein at least one of the ozone generator and the humidifier comprises a sensor, and at least one of the first and second signals comprises sensor information.

103. The method of claim 99 further comprising sending a third signal from at least one of the humidifier and the ozone generator to a communications hub.

104. The method claims 103 further comprising sending a fourth signal from the communication hub a second communication hub of a separate system.

105. The method claim 103 further comprising sending a fifth signal from the communication hub to equipment located in a building remote to the system or in a service vehicle.

106. The method of claim 99 further comprising emitting ozone generating light with a lamp within the ozone generator.

107. The method of claim 99 further comprising sending a sixth signal from one of the humidifier and the ozone generator to a controller or to an individual.

108. The method of claim 99, wherein at least one of the humidifier and the ozone generator comprises a processor and a memory, and the method further comprises operating a local operating system and one or more software applications.
